(19) United States
(12) Patent Application Publication
(10) Pub. No.: US 2015/0003577 A1
(43) Pub. Date: Jan. 1, 2015

(54) METHOD FOR POSITIONING A BODY REGION OF INTEREST IN THE ISOCENTRE OF A CT IMAGING SYSTEM

(71) Applicant: Siemens Aktiengesellschaft, Munich (DE)

(72) Inventors: Peter Aulbach, Forchheim-Kersbach (DE); Ute Feuerlein, Erlangen (DE)

(73) Assignee: Siemens Aktiengesellschaft, Munich (DE)

(21) Appl. No.: 14/378,215

(22) PCT Filed: Jan. 22, 2013
(86) PCT No.: PCT/EP2013/051074

§ 371 Date: Aug. 12, 2014

(30) Foreign Application Priority Data
Feb. 14, 2012 (DE) 10201202165.5

(51) Int. Cl. A61B 6/04; A61B 6/00; A61B 6/03

(52) U.S. Cl. CPC A61B 6/0457; A61B 6/03; A61B 6/4441
USPC 378/9; 378/20

(57) ABSTRACT

A method is disclosed for positioning a body region of interest of a patient in the isocentre of the imaging system of a computer tomograph or C-arm device. The method involves using the computer tomograph or C-arm device to record a topogram of the patient, determining a distance between the body region of interest and the isocentre from the topogram, and shifting a patient positioning device or the imaging system by this distance in order to move the body part into the isocentre. In the proposed method, the topogram is recorded in sections from various directions that are perpendicular to one another. The method enables the body region of interest to be positioned without the use of any external aids.

### PRIORITY STATEMENT

This application is the national phase under 35 U.S.C. §371 of PCT International Application No. PCT/EP2013/051074 which has an International filing date of Jan. 22, 2013, which designated the United States of America, and which claims priority to German patent application number DE 102012202165.5 filed Feb. 14, 2012, the entire contents of each of which are hereby incorporated herein by reference.

### FIELD

At least one embodiment of the present invention generally relates to a method for positioning a body region of interest of a patient in the isocenter of the imaging system of a computer tomograph or C-arm device. In one embodiment, it relates to a method wherein first, a topogram of the patient is recorded with the computer tomograph or C-arm device, and then a distance between the body region of interest and the isocenter is determined from the topogram.

### BACKGROUND

During computed tomography (CT), an imaging system, usually comprising an X-ray tube and an X-ray detector arranged opposite each other, rotates about an axis of rotation in order to obtain radiographs from different projection angles. The point at which the central beam of radiation intersects with the axis of rotation is also referred to here as the isocenter. The CT image is then reconstructed from the radiographs of the individual projections. Prior to recording the CT image, a so-called topogram is often recorded in order to plan the images to be recorded. The topogram provides an overview of the patient and his or her anatomy.

When recording a topogram the imaging system is not rotated, but instead travels along the patient volume only in the axial direction, referred to as the z-direction or the direction of the system axis of the computer tomograph. This produces an overlay image similar to a conventional X-ray image which is used to plan the subsequent X-ray scan for the CT image. The topogram is frequently recorded from the a.p. (anterior to posterior) direction, the p.a. (posterior to anterior) direction, or laterally (from left to right or from right to left respectively), depending on the desired image to be obtained.

For performing CT X-ray imaging with the computer tomograph or a C-arm device, the positioning of the body region of interest in the isocenter of the imaging system is important because the image quality and spatial resolution is best in the center of the measurement field. This is particularly relevant when examining the heart or the skull.

Until now, the height of the patient's position was set with the aid of external positioning lasers attached to the rotating frame of the computer tomograph. A horizontal laser projection on the surface of the patient is used here to align the center line of the patient with the projection. During this process the operator is at the computer tomograph and manually sets the height of the positioning device.

### SUMMARY

At least one embodiment of the present invention is directed to a method for positioning a body region of interest of a patient in the isocenter of the imaging system of a computer tomograph or C-arm device which enables the positioning to be performed without further aids.

A computer tomograph or a corresponding C-arm device designed for carrying out the method is also disclosed. Advantageous refinements of the method and of the computer tomograph or C-arm device are set out in the dependent claims or can be found in the description below and the example embodiment.

With an embodiment of the proposed method, a topogram of the patient is used to position the body region of interest in the isocenter of the imaging system. First a topogram of the patient is recorded using the computer tomograph or C-arm device. For this the topogram is not recorded in the usual way from one direction, but in sections, with two adjacent sections of the topogram being recorded from different directions that are perpendicular to one another. A topogram is thus obtained which shows the different sections of the patient's body from different directions. It is then possible to determine from the topogram a distance between the body region of interest and the isocenter during the recording of the topogram. Either the patient positioning device or the imaging system is then moved by this distance to bring the body region of interest into the isocenter.

An appropriately equipped computer tomograph or an appropriately equipped C-arm device comprises a control and evaluation unit designed in such a way that in one operating mode it records the topogram in accordance with the proposed method, determines the distance between the body region of interest and the isocenter from the topogram, and then activates a positioning control for the patient positioning device or for the imaging system in such a way that the patient positioning device or imaging system is moved a by distance which brings the body part into the isocenter. The body region of interest may either be marked on the topogram by the operator or be identified in the topogram by an algorithm using an appropriate specification.

### BRIEF DESCRIPTION OF THE DRAWINGS

FIG. 1 shows an example of the schematic design of a computer tomograph with which the proposed method may be performed;

FIG. 2 shows a flowchart for carrying out the proposed method; and

FIG. 3 schematically illustrates an example of a topogram in accordance with the proposed method.

### DETAILED DESCRIPTION OF EXAMPLE EMBODIMENTS

With an embodiment of the proposed method, a topogram of the patient is used to position the body region of interest in the isocenter of the imaging system. First a topogram of the patient is recorded using the computer tomograph or C-arm device. For this the topogram is not recorded in the usual way from one direction, but in sections, with two adjacent sections of the topogram being recorded from different directions that are perpendicular to one another. A topogram is thus obtained which shows the different sections of the patient's body from different directions. It is then possible to determine from the topogram a distance between the body region of interest and the isocenter during the recording of the topogram. Either the patient positioning device or the imaging system is then moved by this distance to bring the body region of interest into the isocenter.

With an embodiment of the proposed method, external aids, such as an external laser for example, are no longer required to position the body region of interest in the isocenter of the imaging system. Instead this positioning is performed on the basis of the recorded topogram, which the operator usually requires anyway for planning the subsequent X-ray scan for the CT image. The location of the isocenter is evident from the topogram. For each of the different directions, it lies in the center of the FOV (field of view) (perpendicular to the longitudinal axis of the patient).

In the example embodiment, the two directions of topogram recording are selected so that at least one section of the topogram is recorded in the lateral direction from left to right or from right to left and a further section of the topogram is recorded in the vertical direction. The vertical direction is here understood to refer to the a.p. or p.a. direction with the patient in the conventional supine position. In particular the recording of the topogram from the side (lateral) enables optimal setting of the isocentric position of the patient in the vertical axis. Conversely, recording in the vertical direction enables positioning in the horizontal axis. In the context of computer tomographs or C-arm devices, the vertical axis is also frequently referred to as the x-axis, and the horizontal axis is termed the y-axis. By virtue of recording the topogram in sections from the different directions, the patient dose is much lower than if two conventional topograms were recorded in these directions.

Evaluating the position of the body region of interest can also be automated, so that the positioning of this region in the isocenter can then likewise be performed automatically, that is to say without the intervention of the user, directly by a control unit of the computer tomograph or C-arm device. As a rule this is done by moving the patient positioning device accordingly in the y-direction or by raising or lowering the positioning device in the x-direction. Alternatively, particularly in the case of a C-arm device, it is also possible for the imaging system to be moved by way of motors. In this case the control unit activates the patient positioning device or the imaging system, or their positioning controls respectively.

By virtue of the different recording directions, in the case of whole-body CT scans the proposed method can also automatically determine the height of the surface of the patient positioning device from the topogram and use it to ensure optimal isocentric positioning of the body region of interest. In order to minimize the dose, in this case two complementary topograms are not recorded, but rather recording switches back and forth between a.p./p.a. topogram portions and lateral portions (height information) in each case.

The method may be employed particularly advantageously with “dual source CT systems” which have two imaging units offset at a 90° angle from one another. With such dual source systems, the respective topogram sections from the different directions can then be recorded in each case simply by switching over from the one to the other imaging unit while the patient is being simultaneously moved in the z-direction.

The topogram is preferably composed of a plurality of lateral portions (areas with recording in the lateral direction) and a plurality of vertical portions (areas with recording in the vertical direction) that alternate with one another. The lateral areas serve to extract the relevant information about the optimal vertical positioning height (x-axis) which is especially difficult for the operator to estimate. With certain organs, such as the heart or brain for example, isocentric positioning is required to achieve a high image quality. The sections with lateral recording are therefore expediently selected so that they include the heart and/or the head of the patient.

Switching over between the different directions can either be determined in advance irrespective of the patient's anatomy or can be controlled by a suitable algorithm while the topogram is being recorded. For instance, in one embodiment it is possible to switch over in each case after the patient positioning device has been moved by a given distance in the z-direction. In another embodiment, known algorithms are used which automatically identify the anatomy and organ boundaries from the topogram already while the topogram is being recorded. The algorithms then forward their results, usually segmenting results, continuously and in real time to the control processor of the CT device. The latter records the z-axis values determined for the organ boundaries and switches between the two directions (with a short latency) as indicated by a specification. The specification by the operator indicates the organs or anatomical areas at which a switchover is to be made. If a dual-energy device is used, the switchover can be performed simply by switching between the two mutually offset imaging units. It is however also possible to activate a conventional CT device having only one image recording unit in such a way that the imaging unit rotates through an angle of 90° in each case in order to switch between the different directions.

An appropriately equipped computer tomograph or an appropriately equipped C-arm device comprises a control and evaluation unit designed in such a way that in one operating mode it records the topogram in accordance with the proposed method, determines the distance between the body region of interest and the isocenter from the topogram, and then activates a positioning control for the patient positioning device or for the imaging system in such a way that the patient positioning device or imaging system is moved a by distance which brings the body part into the isocenter. The body region of interest may either be marked on the topogram by the operator or be identified in the topogram by an algorithm using an appropriate specification.

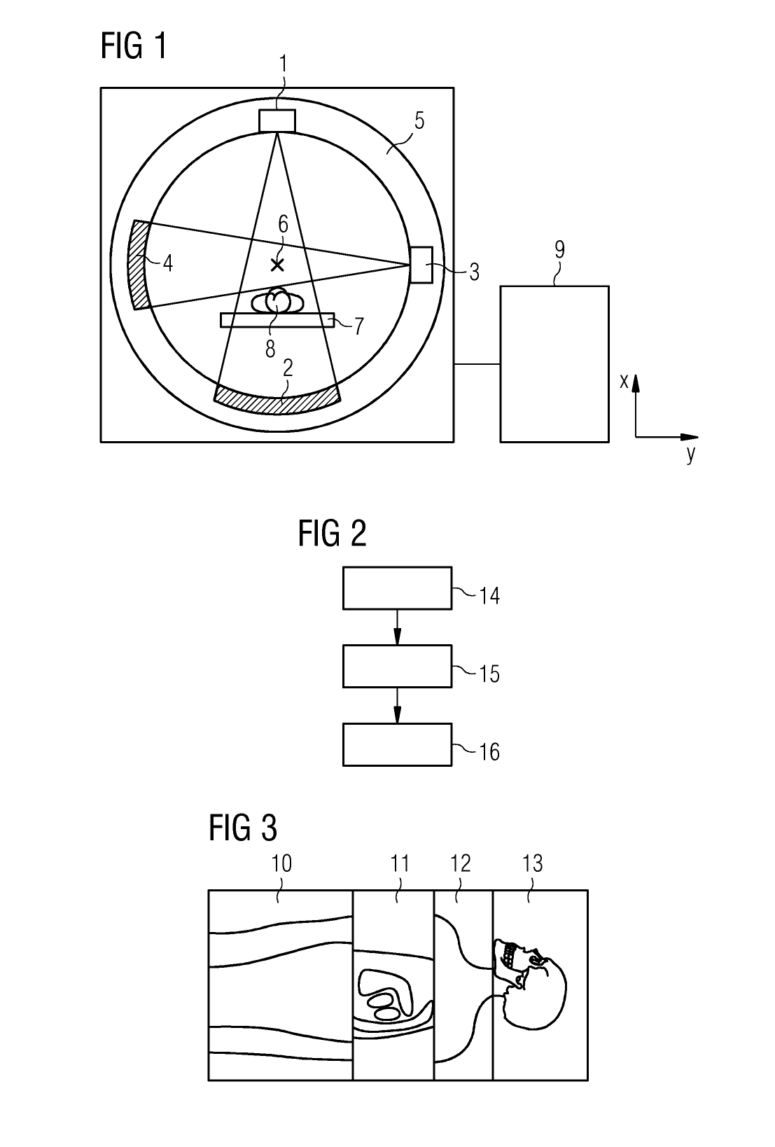

FIG. 1 is a highly schematic diagram of a dual-source computer tomograph which has two image recording units formed in each case by an X-ray source 1, 3 and an X-ray detector 2, 4 opposite the X-ray source. The X-ray sources 1, 3 and X-ray detectors 2, 4 are arranged on a rotating frame 5 that can rotate around the isocenter 6. A patient positioning table 7 is arranged in the examination volume between the X-ray sources 1, 3 and X-ray detectors 2, 4; the table is moved in the direction of the system axis or z-axis (in the example shown in FIG. 1 perpendicular to the plane of the sheet) while the image is being recorded. In FIG. 1 a patient 8 is lying on the patient positioning table 7. The patient positioning table 7 can also be moved vertically (x-direction) and laterally (y-direction) by motors in order to bring the body region of interest of the patient into the isocenter 6.

The computer tomograph is activated by the control and evaluation unit 9 to take radiographs. The measurement data are also evaluated in the control and evaluation unit 9. The control and evaluation unit 9 may of course also be formed by multiple separate units.

The proposed method and the associated imaging device are briefly described again below in relation to an example embodiment with reference to the drawings, in which:

In FIG. 1 it can be seen that the patient's body 8 is currently not in the isocenter 6 of the imaging system. To bring this patient 8, in particular a body region of interest for the subsequent image recording, into the isocenter 6, with the proposed method a topogram of the patient's body, or a part thereof, is first recorded. In the present example this is done by recording a topogram first with the stationary first imaging unit 1, 2 from the a.p. or p.a. direction in a first region, then switching over to image recording by the second imaging unit 3, 4 during the further movement of the patient positioning table 7 in the z-direction in order to obtain a lateral or sagittal image section. Recording then switches to the first imaging unit 1, 2 again, and then to the second imaging unit 3, 4 again. This produces a topogram as schematically illustrated in FIG. 3. The topogram shows a first a.p./p.a. section 10, followed by a first sagittal section 11, a second a.p./p.a. section 12 and a second sagittal section 13. In this example the switchover between the two directions was performed in such a way that both the heart and the skull of the patient were recorded in the lateral direction in order to enable them to be positioned as precisely as possible in the isocenter 6 by adjusting the height of the patient positioning table 7.

Controlling the recording of such a combi-topogram as well as evaluating the topogram can be performed by suitable algorithms implemented in the control and evaluation unit 9. After determining the distance between the body region of interest and the isocenter 6, in the present example the patient positioning table 7 is activated by the control and evaluation unit 9 in such a way that it is moved by this distance in order to bring the body region of interest, for example the heart of the patient 8, into the isocenter 6 of the imaging system.

FIG. 2 shows an again highly schematic flowchart of the individual steps in the proposed method from the recording of the combi-topogram 14, the determination of the distance to the isocenter 15, to the activation of the patient positioning table 16.

Although the invention was illustrated and described in detail with reference to the preferred example embodiment, the invention is not restricted to the examples disclosed. A person skilled in the art may derive other variations from the above without departing from the scope of the invention.

1. A method for positioning a body region of interest of a patient in an isocenter of the imaging system of a computer tomograph or C-arm device,
the method comprising:
recording a topogram of the patient in sections using the computer tomograph or C-arm device, two relatively adjacent sections of the topogram being recorded from different directions that are perpendicular to one another;
determining a distance, between the body region of interest and the isocenter, from the topogram; and
relatively moving at least one of a patient positioning device and the imaging system by the determined distance to bring the body region into the isocenter.

2. The method of claim 1, wherein at least one of the sections of the topogram is recorded in a lateral direction and at least one further of the sections of the topogram is recorded in a vertical direction.

3. The method of claim 1, wherein the imaging system of the computer tomograph or C-arm device includes at least two imaging units, each including an X-ray source and X-ray detector mutually offset by an angle of 90°, and wherein a switchover between the at least two imaging units is performed one or more times to record the topogram in sections from the different directions.

4. The method of claim 1, wherein at least one of a heart and a skull of the patient is recorded in a lateral direction.

5. The method of claim 3, wherein the switchover between the different directions is made on the basis of a specification with aid of an algorithm which identifies at least one of an anatomy and organ boundary from already recorded sections of the topogram and sends corresponding data, while the topogram is being recorded, to a controller which controls the switchover according to the specification on the basis of the data sent by the algorithm.

6. A computer tomograph or C-arm device comprising:
an imaging system including at least one X-ray source, and at least one X-ray detector arranged opposite the at least X-ray source, the at least one X-ray source and X-ray detector being relatively rotatable about an isocenter;
a patient positioning device; and
a control and evaluation unit to activate the computer tomograph or the C-arm device to take radiographs,
wherein the control and evaluation unit is designed to, in one operating mode,
first activate the computer tomograph or the C-arm device to record a topogram of a patient lying on the patient positioning device to record the topogram in sections, two relatively adjacent sections of the topogram being recordable from different directions that are perpendicular to one another,
determine a distance, between a body region of interest of the patient and the isocenter, from the topogram, and
subsequently activate a positioning control for the patient positioning device or for the imaging system to relatively move at least one of a patient positioning device and the imaging system by the determined distance to bring the body region into the isocenter.

7. The computer tomograph or C-arm device of claim 6, wherein
the control and evaluation unit is designed to record at least one of the sections of the topogram in a lateral direction and at least one further of the sections of the topogram in a vertical direction.

8. The computer tomograph or C-arm device of claim 6, wherein the imaging system includes at least two imaging units, each including an X-ray source and an X-ray detector mutually offset by an angle of 90°, and wherein the control and evaluation unit is designed to perform a switchover between the at least two imaging units one or more times to record the topogram in sections from the different directions.

9. The computer tomograph or C-arm device of claim 8, wherein the control and evaluation unit is designed to perform the switchover between the different directions on the basis of a specification with the aid of an algorithm which identifies, while the topogram is being recorded, at least one of an anatomy and organ boundary from already recorded sections of the topogram.

10. The method of claim 2, wherein the computer tomograph or C-arm device includes at least two X-ray sources and at least X-ray detectors, mutually offset by an angle of 90°, forming at least two imaging units, and wherein a switchover between the at least two imaging units is performed one or more times to record the topogram in sections from the different directions.

11. The method of claim 2, wherein at least one of a heart and a skull of the patient is recorded in a lateral direction.

12. The method of claim 3, wherein at least one of a heart and a skull of the patient is recorded in a lateral direction.

13. The method of claim 10, wherein at least one of a heart and a skull of the patient is recorded in a lateral direction.

14. The method of claim 10, wherein the switchover between the different directions is made on the basis of a specification with aid of an algorithm which identifies at least one of an anatomy and organ boundary from already recorded sections of the topogram and sends corresponding data, while the topogram is being recorded, to a controller which controls the switchover according to the specification on the basis of the data sent by the algorithm.

15. The method of claim 12, wherein the switchover between the different directions is made on the basis of a specification with aid of an algorithm which identifies at least one of an anatomy and organ boundary from already recorded sections of the topogram and sends corresponding data, while the topogram is being recorded, to a controller which controls the switchover according to the specification on the basis of the data sent by the algorithm.

16. The computer tomograph or C-arm device of claim 7, wherein the imaging system includes at least two imaging units, each including an X-ray source and an X-ray detector mutually offset by an angle of 90°, and wherein the control and evaluation unit is designed to perform a switchover between the at least two imaging units one or more times to record the topogram in sections from the different directions.

17. The computer tomograph or C-arm device of claim 16, wherein the control and evaluation unit is designed to perform the switchover between the different directions on the basis of a specification with the aid of an algorithm which identifies, while the topogram is being recorded, at least one of an anatomy and organ boundary from already recorded sections of the topogram.
